(19) United States
(12) Patent Application Publication
(10) Pub. No.: US 2011/0238153 A1
(43) Pub. Date: Sep. 29, 2011

(54) ENDOPROSTHESES

(75) Inventors: Liliana Atanasoska, Minneapolis, MN (US); Angela Kostrewa, Riverview, FL (US); Rajesh Radhakrishnan, Maple Grove, MN (US); Robert W. Warner, Woodbury, MN (US); Lagiang Tran, Coon Rapids, MN (US); James Lee Shippy, III, Maple Grove, MN (US); Edward E. Parsonage, Saint Paul, MN (US)

(73) Assignee: BOSTON SCIENTIFIC SCIMED, INC., Maple Grove, MN (US)

(21) Appl. No.: 13/069,961

(22) Filed: Mar. 23, 2011

Related U.S. Application Data
(60) Provisional application No. 61/317,836, filed on Mar. 26, 2010.

(51) Int. Cl. A61F 2/82; B05D 3/10; C25F 3/00

(52) U.S. Cl. 623/1.15; 623/1.46; 216/37; 205/640

(57) ABSTRACT

Endoprostheses include an endoprosthesis wall that includes a surface layer that includes a metallic material and that defines a plurality of discrete pores. A porous material is disposed in one or more pores of the surface layer. The endoprostheses can, for example, deliver a therapeutic agent, such as a drug, in a controlled manner over an extended period of time.

### CROSS-REFERENCE TO RELATED APPLICATION

This application claims priority under 35 USC §119(e) to U.S. Provisional Patent Application Ser. No. 61/317,836, filed on Mar. 26, 2010, the entire contents of which are hereby incorporated by reference.

### TECHNICAL FIELD

This invention relates to endoprostheses.

### BACKGROUND

The body includes various passageways such as arteries, other blood vessels and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen. Stent delivery is further discussed in Heath, U.S. Pat. No. 6,290,721.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

### SUMMARY

In one aspect, the invention features an endoprosthesis that includes an endoprosthesis wall with a surface layer that includes a metallic material and that defines a plurality of discrete pores, the discrete pores predominantly having a pore size of 500 nm or more; and a porous material disposed in one or more of the discrete pores of the surface layer.

In another aspect, the invention features an endoprosthesis with an endoprosthesis wall that includes a surface layer that includes a metallic material and that defines a plurality of discrete pores, the discrete pores predominantly having a pore size of 500 nm or more; a porous material disposed in one or more of the discrete pores of the surface layer; and a polymer coating anchored over the surface layer by the porous material.

In another aspect, the invention features an endoprosthesis that includes an endoprosthesis wall with a surface layer that includes a metallic material and that defines a plurality of discrete pores, the discrete pores predominantly having a pore size of 500 nm or more; a porous material disposed in one or more of the discrete pores of the surface layer; and a therapeutic agent impregnated in the porous material in one or more of the discrete pores of the surface layer.

In another aspect, the invention features a method for making an endoprosthesis that includes forming on a surface of an endoprosthesis preform a plurality of discrete pores; depositing a first material into one or more of the discrete pores; corroding the first material to form a second material in the discrete pores; and treating the second material in the discrete pores in a locally high alkaline environment to form a third material.

Embodiments and/or aspects may include any one or more of the following features. The endoprosthesis wall further comprises a non-porous solid material disposed in one or more of the discrete pores. The non-porous solid material comprises a bioresorbable ceramic. The bioresorbable ceramic comprises a material selected from the group consisting of hydroxyapatite, magnesium hydroxide, and calcium hydroxide. The porous magnesium oxide material and the non-porous solid material are disposed in different discrete pores. The porous magnesium oxide material and the non-porous solid material are disposed in the same discrete pores. The non-porous solid material comprises a matrix structure and contains a drug. There is a therapeutic agent in the porous magnesium oxide material. There is a polymer coating covering the surface layer, anchored by the porous magnesium oxide material disposed in one or more of the discrete pores. The magnesium oxide material extends beyond the surface of the well. The porous material has a morphology selected from a group consisting of nano-ribbon, nano-needle, nano-rod, rice grain, corn flakes, and cauliflower. The porous material comprises magnesium oxide or magnesium hydroxide. The porous material comprises a morphology-stabilizing sol gel coating. The sol gel coating comprises MgO—CaO—FeO—ZnO—TaO. The porous magnesium oxide material comprises a hydrophobicity enhancing agent. The hydrophobicity enhancing agent comprises an Oleic or Stearic acid. The metallic material is selected from a group consisting of stainless steel, Co—Cr alloy, MP35N, Nitinol, and PERSS. The polymer coating includes a therapeutic agent. The polymer is bioerodible. The endoprosthesis wall comprises an abluminal surface region and a luminal surface region, and the surface layer is in the abluminal surface region. The pore width is 500 nm or more. The oxide extends by about 0.1 micron or more above surface.

Embodiments and/or aspects may include any one or more of the following features. The first material is corroded by applying an electrolyte to the first material. The electrolyte applied to the first material comprises an aqueous NaCl solution or a solid polymer electrolyte. The first material is corroded by anodically dissolving the first material. The first material is anodically dissolved by applying a positive voltage to the first material. The second material is treated by a water-alcohol solution. The third material is coated with a morphology-stabilizing sol gel coating. A surface-modifying material comprising an oleic or stearic acid is loaded to the third material. A radiopaque material is added into the third material. The first material comprises magnesium or magnesium alloy. The second material comprises magnesium salt, magnesium chloride, magnesium sulfate, or a combination thereof. The third material comprises magnesium oxide or magnesium hydroxide. A solid non-porous material is deposited into one or more of the pores in which the first material is not deposited. A solid non-porous material is deposited into one or more of the pores in which the first material is deposited.

Embodiments and/or aspects may include any one or more of the following advantages. A stent can be provided having enhanced adhesion of a polymer coating to a stent surface. A stent can also be provided that delivers a drug without the use of a polymer coating on the stent surface. A stent can also be provided that delivers a drug with a polymer conjugate through discrete sites on the stent surface. The stent surface can be treated to form a plurality of discrete pores which are subsequently filled by a porous material. For example, the metal surface of a stent can be treated by laser ablation to create relatively large, discrete pores, e.g. more than 500 nm in cross-section. The porous material is formed in the discrete pores by first forming a thin layer of biodegradable metal, e.g. magnesium, in the discrete pores and next corroding the biodegradable metal to form the porous material, e.g. magnesium hydroxide. Portions of the porous material in the discrete pores can extend beyond the stent surface and act as discrete anchors for additional coatings, e.g. a polymer coating. A polymer coating containing a drug can be anchored, e.g. by the porous material, with enhanced adhesion on the stent surface. The porous material can have a nano-structured morphology, e.g. rice grain morphology, nano-rod morphology, or nano-needle morphology. The nano-structured porous material in the discrete pores can have high surface areas and absorb drugs. The discrete pores with porous material can act as a drug reservoir, and the porous morphology of the porous material can meter the delivery of the drug from the reservoir. In embodiments, the stent is free of any non-therapeutic polymer, such as a polymer carrier for a drug. In other embodiments, a polymer-drug conjugate can be loaded to the porous material.

All publications, patent applications, patents, and other references mentioned herein are incorporated by reference herein in their entirety.

Other features and advantages of the invention will be apparent from the following detailed description, and from the claims.

### DESCRIPTION OF DRAWINGS

FIGS. 1A-1C are longitudinal cross-sectional views, illustrating delivery of a stent in a collapsed state, expansion of the stent, and deployment of the stent.

FIG. 2 is a perspective view of a fenestrated stent.

FIG. 3 is a cross sectional view of a stent wall along AA′ in FIG. 2.

FIGS. 4A-4F are enlarged plan views (photographs) of a part of a stent wall surface in FIG. 2.

FIG. 5 is a cross sectional view of a stent wall of FIG. 3 with porous material in discrete pores anchoring a polymer coating.

FIG. 6 is a cross sectional view of a stent wall of FIG. 3 with porous material in discrete pores including therapeutic agents.

FIG. 7 is a cross sectional view of a stent wall of FIG. 3 with porous material in discrete pores including polymers that include therapeutic agents.

FIG. 8A-8B are two cross sectional views of stent walls.

FIGS. 9A-9E are generally diagrammatic representations of a method for making a stent.

Like reference symbols in the various drawings indicate like elements.

### DETAILED DESCRIPTION

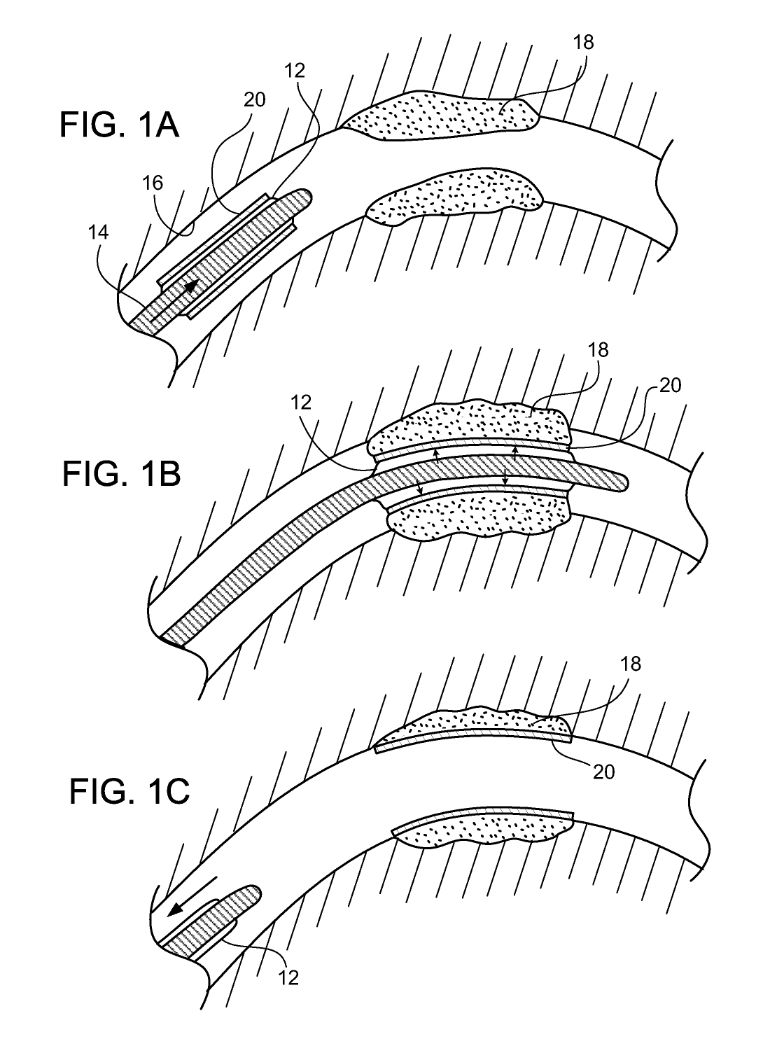

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

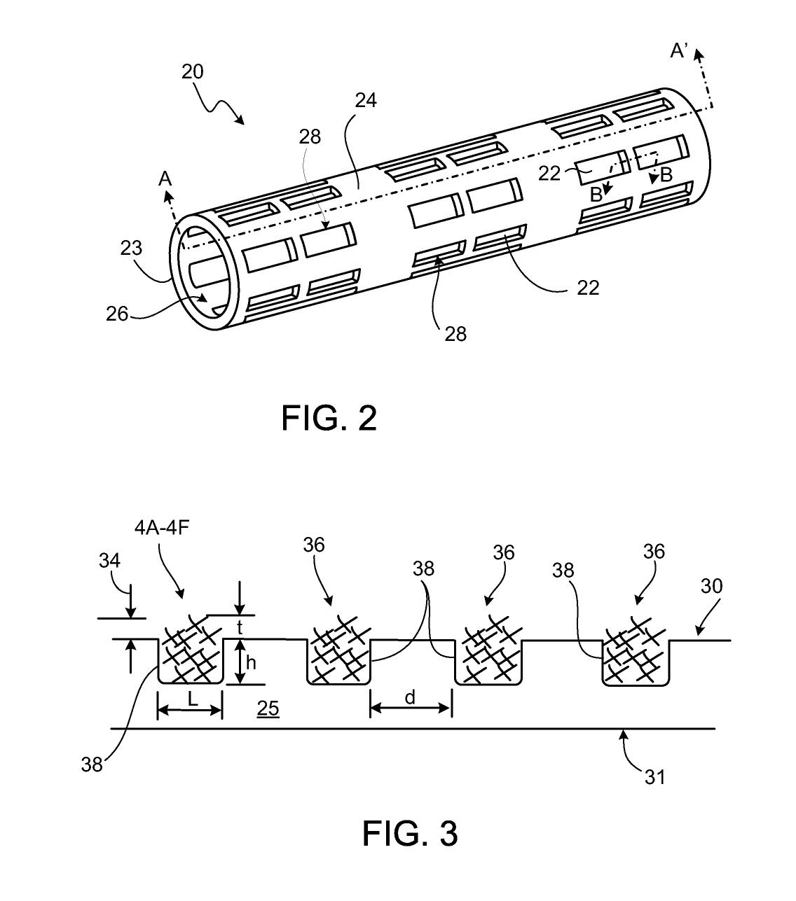

Referring to FIG. 2, stent 20 includes a plurality of fenestrations 22 defined in a wall 23. Stent 20 includes several surface regions, including an outer, or abluminal, surface 24, an inner, luminal (or adluminal), surface 26, and a plurality of cutface surfaces 28. The stent can be balloon expandable, as illustrated above, or a self-expanding stent. Examples of stents are described in Heath '721, supra.

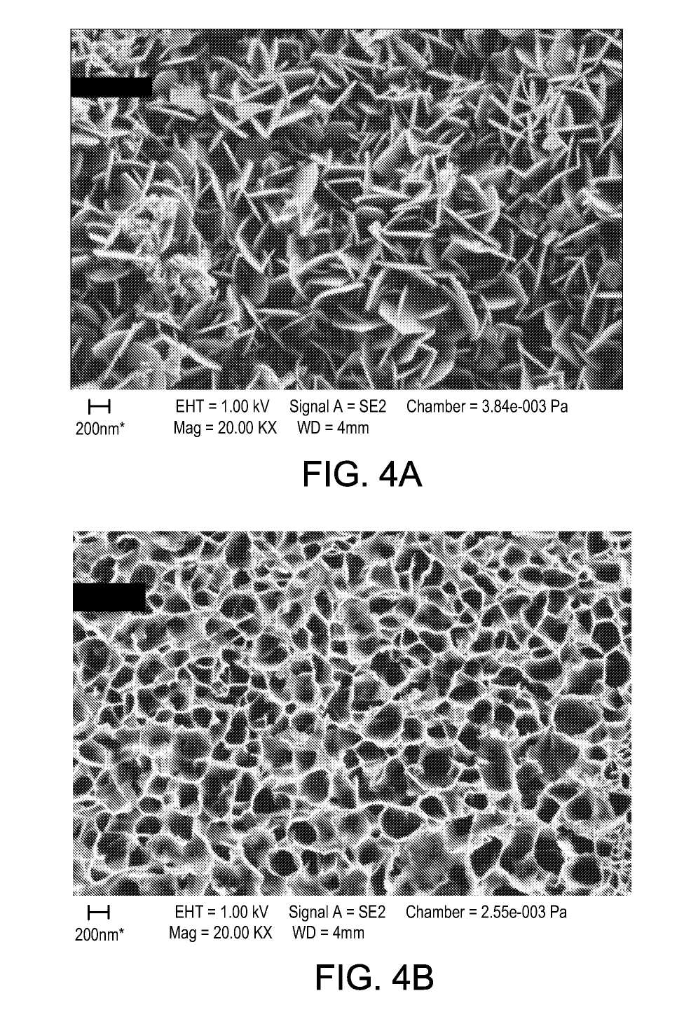

Referring to FIG. 3, a cross-sectional view, a stent wall 23 includes a substrate or a stent body 25 with an abluminal surface region 30 and a luminal surface region 31, a plurality of discrete pores 38 with a porous material 36 disposed in one surface region, e.g. abluminal surface region 30, and a tie layer 34 having discrete anchoring sites on surface region 30.

In embodiments, stent body 25 is formed, e.g., of a metallic material such as a metal, e.g. a metal alloy. Examples of the metallic material include 316L stainless steel, Co—Cr alloy, Nitinol, PERSS, MP35N, and other suitable metallic materials. Discrete pores 38 have an average depth h, width L, and the distance between neighboring pores is d. In embodiments, the average depth h of discrete pores 38 is about 0.5 microns to about 50 microns (e.g., about 0.5 microns to about 30 microns or about 0.5 microns to about 20 microns), and the average width L of discrete pores 38 is at least 500 nm (e.g., at least about 800 nm or at least about 1000 nm). In embodiments, the smallest distance d between neighboring pores is at least about 30 microns (e.g., about 50 microns to about 100 microns).

In embodiments, porous material 36 is a ceramic. In particular embodiments, porous material includes a magnesium oxide or iron oxide based ceramic, e.g. MgₓO_{y}, Mg(OH)₂, FeₓO_{y}, Fe(OH)ₓ, mixed FeOₓ and MgOₓ, calcium oxides, such as CaO or Ca(OH)₂, zinc oxides, such as ZnO, or manganese oxides, such as MnOₓ. Generally, the porosity of porous material 36 is highly tailorable. For example, the pore density of porous material 36 can be controlled by varying conditions of forming these materials in pores 38. Porous material 36 can have a high surface area and thus facilitate drug adsorption to and release from the stent body. In embodiments, porous material 36 fills pores 38 and extends beyond surface 30 by an average height t. In embodiments, the average height t is about 0.1 micron to about 50 microns (e.g., about 0.1 micron to about 30 microns or about 0.1 micron to about 10 microns). Extending portions of porous material 36, e.g. beyond surface 30, forms a tie layer 34 with discrete anchoring sites disposed about surface 30. Tie layer 34 can effectively anchor a coating, e.g. a polymer coating, over surface 30 by enhancing the adhesion between surface 30 and a coating on surface 30. Further, surface 30 having a tie layer with discrete anchoring sites can enhance adhesion more than a surface with a continuous tie layer, e.g. a surface that is uniformly roughened.

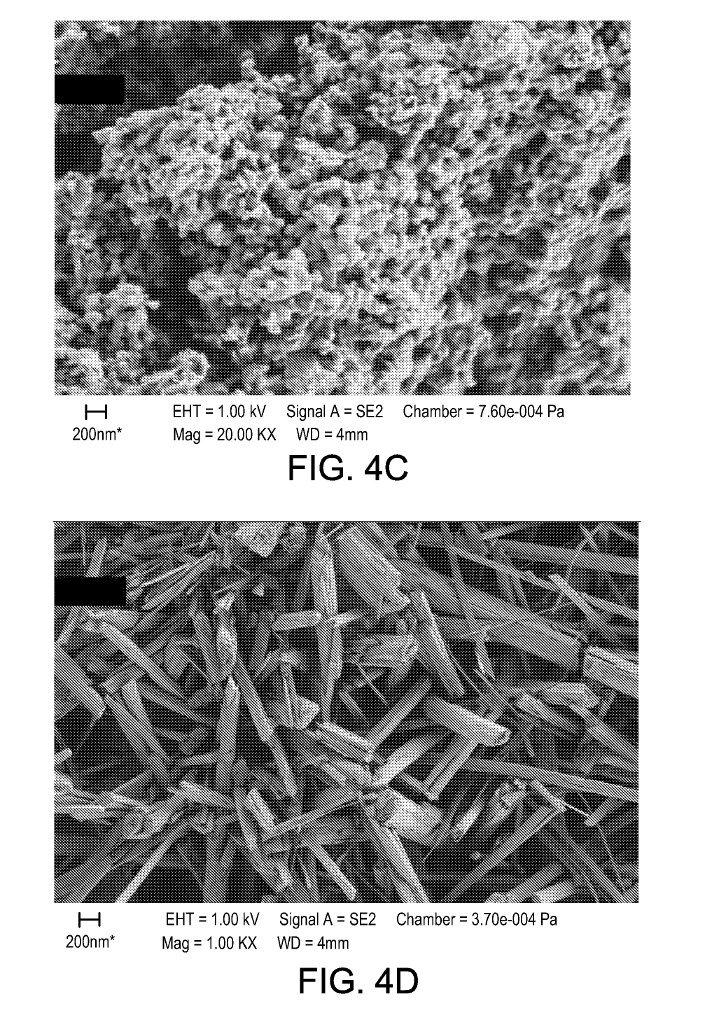

In embodiments, porous material 36 has a nano-structured morphology. Referring to FIGS. 4A-4F, six exemplary nano-structured morphologies of porous material 36 are shown. Referring particularly to FIG. 4A, the porous material can have a morphology characterized by defined rice grains and high roughness. Referring particularly to FIG. 4B, the porous material can have a morphology characterized by defined corn flakes. Referring particularly to FIG. 4C, the porous material can have a morphology characterized by defined cauliflowers. Referring particularly to FIG. 4D, the porous material can have a morphology characterized by defined by nano-rods. Referring particularly to FIG. 4E, the porous material can have a defined nano-ribbon morphology. Referring to FIG. 4F, the porous material can have a morphology characterized by defined nano-needles. The morphology of the porous material can be tuned by adjusting various parameters during the preparation processes. In embodiments, porous material 36 in pores 38 has the same morphology, e.g. rice grain morphology. In other embodiments, porous material 36 in different pores in different regions of stent surface 30 has different morphologies.

In some embodiments, a sol gel coating, e.g. MgO—CaO—FeO—ZnO—TaO or manganese oxide, can be formed on top of nano-structured porous material 36 in pores 38 (not shown in figures). The coating can, e.g., stabilize the formed morphologies. Other coatings or methods can also be used. For example, cathodic electro-deposition for oxide formation and sol-electrophoresis can be used. Phosphate coatings that can be formed by wet chemistry and/or nitride coatings formed by cathodic electrodeposition or chemical/physical vapor deposition. The formed morphologies can also be stabilized using colloidal stabilization. Examples of colloidal stabilization is described in Studart et al., “Colloidal Stabilization of Nanoparticles in Concentrated Suspensions”, Langmuir, Vol. 23, pp. 1081-1090, 2007.

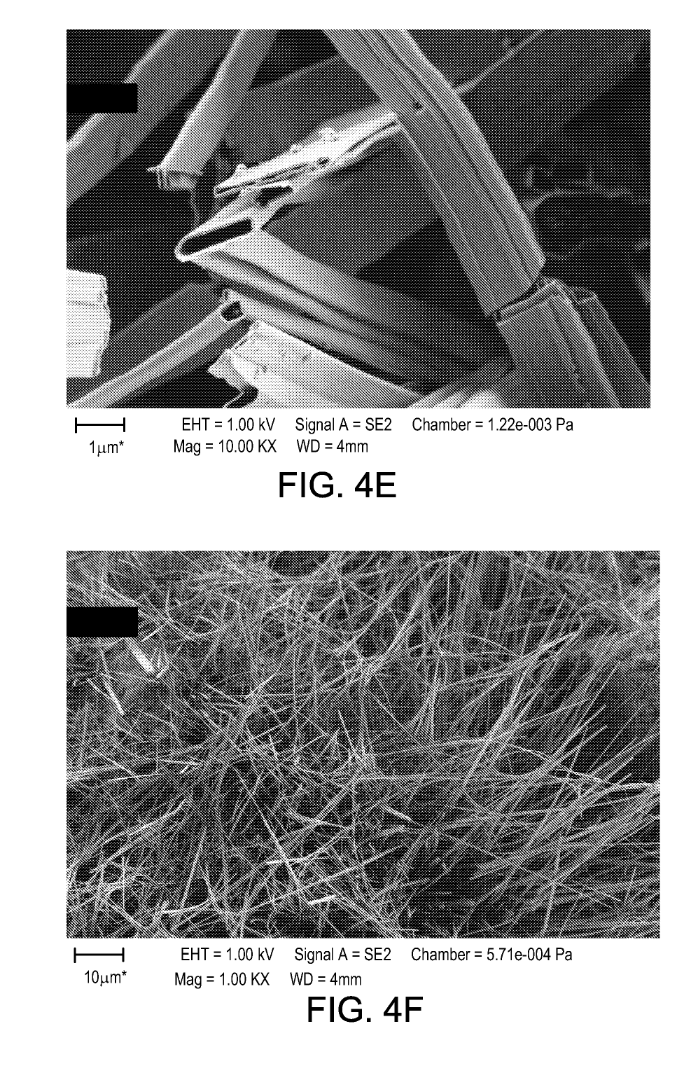

Turning to FIG. 5, a cross-sectional view, a stent wall 50 includes a stent body 25, made of a metallic material, e.g. a metal or metal alloy, with discrete pores 38 disposed over luminal surface 30, where the pores are filled with porous material 36 as is shown in FIG. 3, and a polymer coating 51 anchored to luminal surface 30 through tie layer 34. The adhesion between layer 51 and stent body 25 is greatly enhanced through the discrete anchoring sites created by porous material 36 about surface 30. In particular embodiments, the nano-structured porous material has nano-ribbon or nano-needle morphology. Beneficial effect of discontinuity on adhesion enhancement is also discussed in Gorb et al., Proceeding of the Royal Society, London Series B 267, 1239-1244 and Chung et al., J. R. Soc. Interface 2, 55 (2005). In embodiments, polymer coating 51 includes a therapeutic agent, for example, a drug, e.g. paclitaxel or everolimus.

In embodiments, polymer coating 51 includes a bioresorbable material. For example, polymer coating 51 includes a biodegradable copolymer, such as poly(lactic-co-glycolic acid) (PLGA), a aliphatic polyester, such as polylactide (PLA), or a biodegradable polyester, such as polycaprolactone (PCL). Other polymers can also include, for example, styrene isoprene butadiene (SIBS), ethylene vinyl acetate, poly butyl methacrylate, phosphoryl coline based polymer, polyvinylidene fluoride copolymers, and lactic acid based polymers. In embodiments, the bioresorbable material in polymer coating erodes in the body or body lumen by, e.g., surface erosion processes. In embodiments, the polymer is biostable. In embodiments, polymer coating 51 can have a thickness of about 100 nm to about 2 microns (e.g., about 300 nm to about 2 microns or about 500 nm to about 2 microns). Other suitable polymers and drugs can also be used. Biodegradable polymer is also described in Lakshmi et al., “Biodegradable polymers as biomaterials”. Prog. Polym. Sci. Vol. 32, pp. 762-798, 2007.

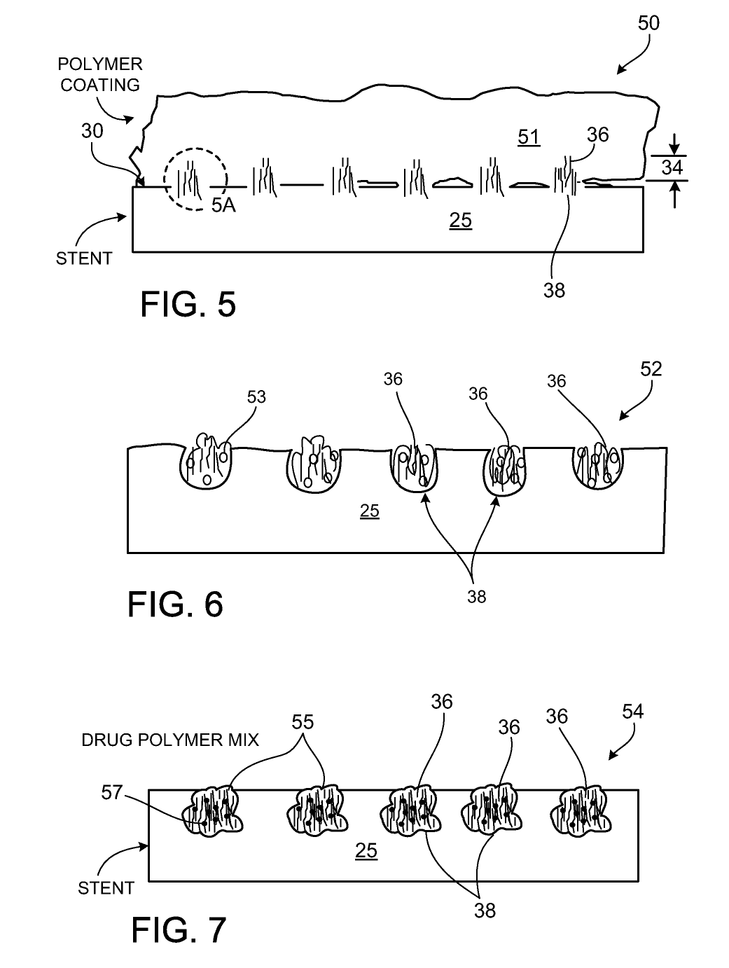

Referring to FIG. 6, a cross-sectional view, a stent wall 52 includes a stent body 25, made of a metallic material, e.g. a metal or metal alloy, with discrete pores 38 disposed over luminal surface 30, where the pores are filled with porous material 36, e.g. as shown in FIG. 3, and a drug 53 is impregnated in the pores of porous material 36. In particular embodiments, porous material 36 has a rice grain, corn flakes, or cauliflower morphology. The nano-sized pores in porous material 36 with these particular morphologies are impregnated with a drug and meter the release of the loaded drug into the lumen. In embodiments, stent wall 52 provides a polymer free drug delivery system that is devoid of complicated multi-layer coating. To provide a long-term therapeutic benefit after the endoprosthesis is implanted in a human body, release of the one or more therapeutic agents at a therapeutic level over an extended period of time is desirable. Generally, a large volume of the therapeutic agent or blend of therapeutic agents utilized is stored in the stent, and the release rate is controlled by the properties of the porous material, as described herein. Generally, pores 38 and the high surface areas of porous material 36 in pores 38 can provide reservoirs for therapeutic agents, for example anti-restenosis drugs, such as paclitaxel and everolimus, and nano-structured porous feature of porous material 36 can provide nano-sized channels to meter the releasing of the therapeutic agents stored in the reservoirs. The reservoirs and the channels provide a long-term drug eluting and sustainable therapeutic effect after the stent is delivered to lumen. Generally, different nano-structured morphologies of porous material 36 can provide different drug delivery and release profiles. In embodiments, porous material 36 can be made to be hydrophobic by surface modification of the material with an oleic or stearic acid. Scheme 1 (below) shows surface modification of magnesium hydroxide with a stearic acid. In embodiments, hydrophobic nano-structured porous material can have improved adsorption of hydrophobic therapeutic agents, such as paclitaxel.

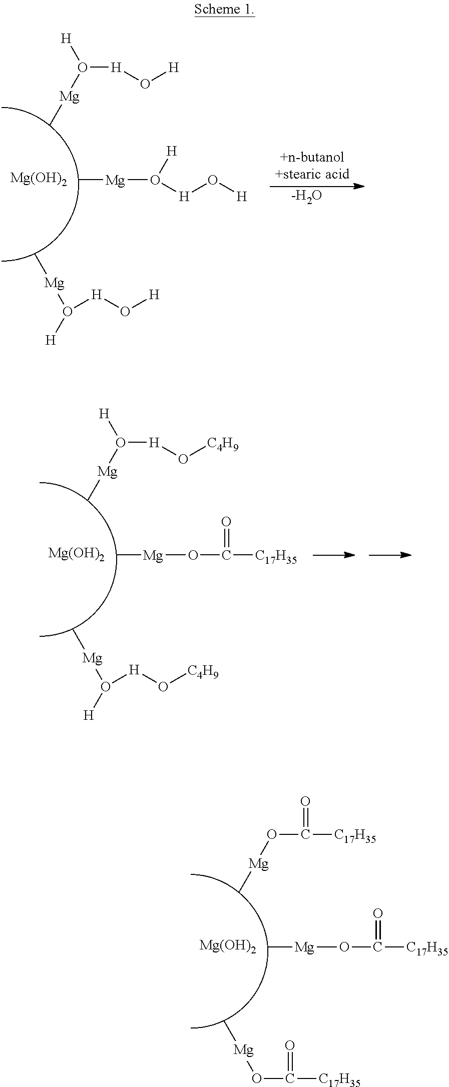

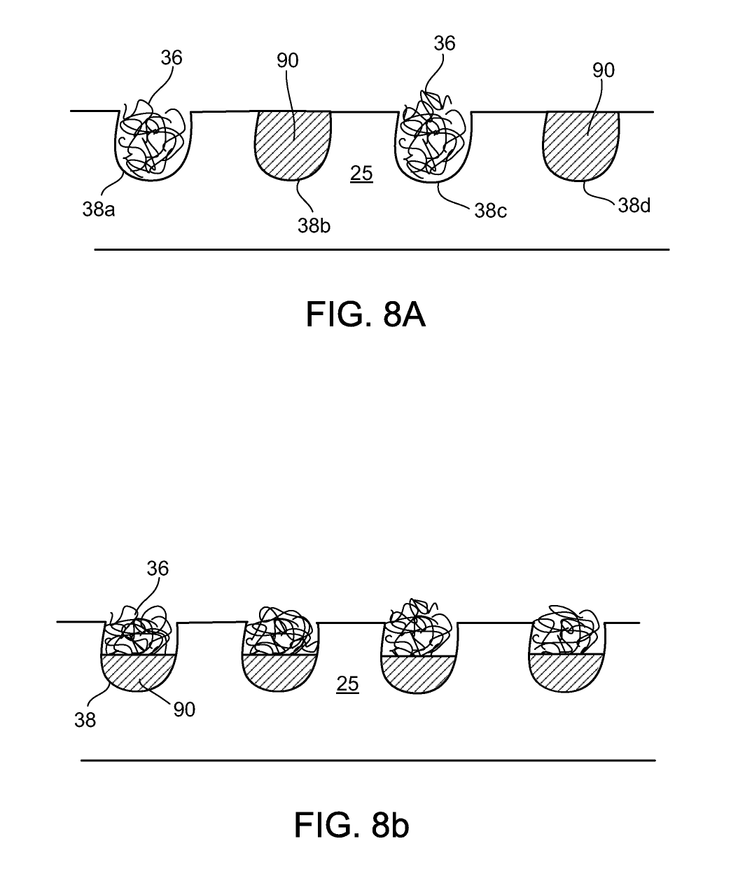

Referring to FIG. 7, a cross-sectional view, a stent wall 54 includes a stent body 25, made of a metallic material, e.g. a metal or metal alloy, with discrete pores 38 disposed over luminal surface 30, where the pores contain porous material 36, e.g. as shown in FIG. 3, and a polymer 55 is loaded into pores 38. In embodiments, polymer 55 includes an anti-restenosis drug 57, e.g. paclitaxel or everolimus. In particular embodiments, the anti-restenosis drug is in a conjugated form, e.g. paclitaxel-polyglutamic acid conjugate.

In embodiments, polymer 55 is tightly bound to porous material 36 through the nano-structured morphologies, e.g. rice grain or cauliflower, of the porous material.

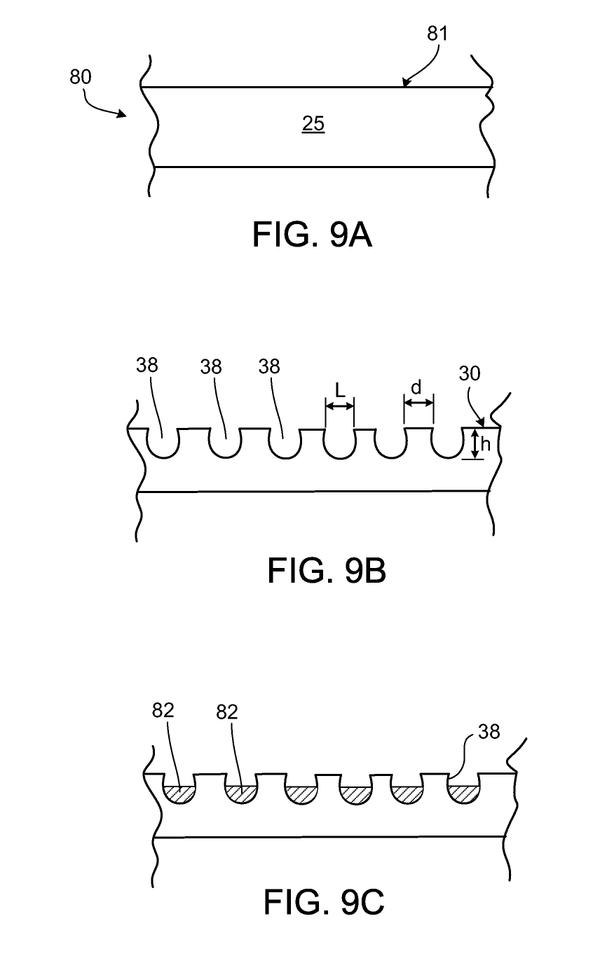

In some embodiments, the material filling the pores 38 of FIGS. 3 and 5-7 can be at least partially solid instead of being porous. In the example shown in FIG. 8A, some of the pores 38, e.g., pores 38a and 38c, are filled with the porous material 36 having morphology as described above and loaded with a drug. Some of the pores 38, e.g., pores 38b and 38c, are filled with a solid material 90 loaded with a drug. The solid material can be a bio-resorbable ceramic including, for example, hydroxyapatite, and minerals susceptible to bioresorption such as magnesium hydroxide, and calcium hydroxide. The solid material 90 forms a matrix that encapsulates the drug that can be resorbed more slowly than those included in the porous material 36. Thus the drug encapsulated in the solid material 90 can elute for a long period of time when in contact with a body fluid. The slow resorption rate of the solid material provides a long-term drug eluting profile for the stent. The combination of the porous material 36 and the solid material 90 loaded with drugs allows the drug to initially elute from the porous material 36 at a desired rate after the stent is delivered, and later constantly elute from the solid material 90. A combined short-term and long-term drug eluting profile can be chosen by, for example, selecting the locations and numbers of the pores 38 to be filled with the porous materials 36 and the solid materials 90.

In the example shown in FIG. 8B, each pore 38 of FIGS. 3 and 5-7 can be filled with a combination of the porous bioresorbable material 36 and the solid bioresorbable material 90. A drug can be incorporated into the porous material 36 and the solid material 90. In some embodiments, one or more different drugs can be incorporated in the two materials 36, 90. The arrangement of the two drug containing materials 36, 90 can be adjusted to provide a desired drug eluting profile. For example, the upper half of each pore 38 can be filled with the drug containing porous material 36 and the lower half of the pore 38 can be filled with the drug containing solid material 90. When in contact with a body fluid, the drug in the porous material 36 elutes initially with the degradation of the porous material 36. Then the solid material 90 is exposed to the body fluid to provide a long term drug elution. Other arrangements of the two materials 36, 90 can be applied. Different pores 38 can include different arrangements of the two materials. The overall drug eluting profile can be adjusted, for example, by controlling the amount and location of the porous material 36 relative to those of the solid material 90.

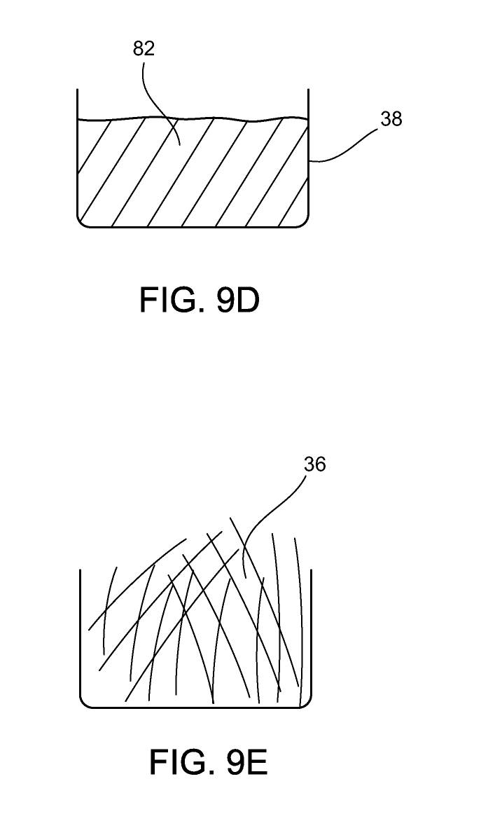

Referring to FIGS. 9A-9E, an exemplary process for making a stent is illustrated. To make a stent exemplified in FIG. 3, a stent preform, such as a metal tube, that includes a metallic material and has a surface defining a plurality of discrete pores is provided. For example, the stent preform with discrete pores can be provided by creating pores on a non-porous stent preform. Next, each discrete pore is coated with a thin layer of a bioresorbable metal or metal alloy, e.g. Mg or Mg alloy. Finally, the bioresorbable films are locally corroded to form in the discrete pores a nano-structured porous material, e.g. magnesium hydroxide or magnesium oxide, that includes various morphologies as shown in FIGS. 4A-4F.

Referring particularly to FIGS. 9A and 9B, discrete pores 38 are created on the non-porous stent preform 80, e.g., made of stainless steel, such that surface 30 (e.g., an abluminal surface) defines a plurality of discrete pores 38. In embodiments, discrete pores 38 are made by a micro hole drilling process with selective masking. For example, stent surface 30 can be covered by a mesh mask, and then a laser can be applied to the masked surface to create the plurality of discrete pores. The shape, width, L, and depth, h, of the pores, and the spacing or distance, d, between the closest-spaced pores can be adjusted by selecting masks with desired shapes and sizes and/or by controlling the various parameters, such as energy and/or frequency of the laser pulses, related to laser ablation.

Referring now to FIGS. 9C and 9D, a thin layer of biodegradable metallic material 82, e.g. a magnesium metal or magnesium alloy, is deposited in pores 38. For example, metallic material 82 can be deposited by physical vapor deposition, e.g. vacuum evaporation or pulsed laser deposition, electrodeposition, ion implantation, or melt deposition and extrusion. In embodiments, biodegradable metallic material 82 can be selectively formed inside pores 38 by applying a mask to the porous surface 30 prior to the deposition. The properties of the layer, e.g. thickness or mass-density, formed by material 82 in pores 38 can be tuned by choosing different deposition methods and/or by tuning the various conditions for deposition.

Referring now to FIG. 9E, porous material 36 is formed by corroding biodegradable metallic material 82. First, material 82, such as magnesium, is exposed to an environment that induces and accelerates corrosion by, for example, local application of an electrolyte, such as an aqueous NaCl solution or a solid electrolyte, to material 82. Material 82 is then corroded by a combined chloride anion and galvanic effect. Alternatively, material 82 can be dissolved anodically by applying a positive voltage to the stent body 25 that includes metallic material 82. As a result of the corrosion or the anodic dissolution, high concentration, e.g., of magnesium salts, chlorides, or sulfates, depending on the electrolyte used for corrosion or anodic dissolution, are formed in the discrete pores. Next, porous material 36 that includes magnesium oxides or magnesium hydroxides is formed by treating the formed magnesium salts, chlorides, or sulfates in a locally highly alkaline environment. Porous material 36 can be produced with different nano-structured morphologies that are exemplified in FIGS. 4A-4F by controlling conditions such as duration of anodic dissolution/corrosion, electrolyte concentration and formulation, pH, and reaction temperature.

In particular embodiments, magnesium hydroxide can be formed by applying a solution chemical process, e.g. homogeneous precipitation, to the magnesium salts, chlorides, or sulfates in the presence of complex water-soluble polymer dispersants. In embodiments, the magnesium salts, chlorides, or sulfates can serve as a magnesium precursor, and NH₃ and NaOH aqueous alkaline solution can be used as precipitators. The alkaline solution is injected into discrete pores 38 by a peristaltic pump at variable speeds, where precipitation reaction takes place and porous material 36 composed of a magnesium hydroxide is formed. In embodiments, dispersants are added so that the shape and size of the nano-structured morphologies of the so-formed porous material 36 can be controlled. In some embodiments, tailored-made dispersants with head-tail architecture can be used for preparation of an aqueous suspension containing ceramic nanoparticles. The dispersants can include a head group that can efficiently adsorb onto the metal oxide surface. The dispersants can also include a tail group that includes a soluble (e.g., water-soluble) polymer or oligomer and that extends towards the suspension aqueous phase. The pH and temperature of the solution are monitored during the precipitation reaction. Different morphologies can be formed by controlling processing condition.

In embodiments, other morphologies can be prepared by varying conditions of preparation such as reaction temperature and/or injection speed of the alkaline solution. Control of morphological structure is further described in attorney docket No. 10527-859001, filed contemporaneously herewith, Yan et al., Nanotechnology 15, 1625 (2004), Lv et al., Nanotechnology 15, 1576 (2004), and Guo et al., Electrochimica Acta 52, 2570 (2007). Brucite microstructures are described in Kogure et al., “Microstructure of nemalite, fibrous iron-bearing brucite”, Mineralogical Journal, Vol. 20, No. 3, pp. 127-133, July 1998; Liebling et al., “Optical Properties of Fibrous Brucite from Asbestos, Quebec”, American Mineralogist, Vol. 57, pp. 857-864, 1972; Buster et al., “Crystal Habits of the Magnesium Hydroxide Mineral Bructite Within Coral Skeletons”, poster, 2006; and Hahn et al., “A novel approach for the formation of Mg(OH)₂/MgO nanowhiskers on magnesium: Rapid anodization in chloride containing solutions”, Electrochemistry Communications, Vol. 10, pp. 288-292, 2008. Corrosion of metals is also described in Matijević, “Colloid Chemical Aspects of Corrosion of Metals”, Pure & Appl. Chem., Vol. 52, pp. 1179-1193, 1980 and Antunes et al., “Characterization of Corrosion Products Formed on Steels in The First Months of Atmospheric Exposure”, Materia, Vol. 8, No. 1, pp. 27-34, 2003. Electrodeposition is described in Zou et al., “Highly textural lamellar mesostructured magnesium hydroxide via a cathodic electrodeposition process”, Materials Letters, Vol. 61, pp. 1990-1993, 2007; Park et al., “Cathodic electrodeposition of RuO₂ thin films from Ru(III)Cl₃ solution”, Materials Chemistry and Physics, Vol. 87, pp. 59-66, 2004; and Lee et al., “A study on electrophoretic deposition of Ni nanoparticles on pitted Ni alloy 600 with surface fractality”, Journal of Colloid and Interface Science, Vol. 308, pp. 413-420, 2007. Descriptions of coating morphology and method of making are also provided in Yang et al., “Solution phase synthesis of magnesium hydroxide sulfate hydrate nanoribbons”, Nanotechology, Vol. 15, pp. 1625-1627, 2004; Guo et al. “Investigation of corrosion behaviors of Mg-6Gd-3Y-0.4Zr alloy in NaCl aqueous solutions”, Electrochemica Acta, Vol. 52, pp. 2570-2579, 2007; Mobedi et al., “Studying the Degradation of Poly(L-lactide) in Presence of Magnesium Hydroxide”, Iranian Polymer Journal, Vol. 15, No. 1, pp. 31-39, 2006; Li et al., “A novel method for preparing surface-modified Mg(OH)₂ nanocrystallines”, Materials Science and Engineering A 452-453, pp. 302-305, 2007; Lv et al., “In situ synthesis of nanolamellas of hydrophobic magnesium hydroxide”, Colloids and Surfaces A: Physiochem. Eng. Aspects, Vol. 296, pp. 97-103, 2007; Lv et al., “Controlled growth of three morphological structures of magnesium hydroxide nanoparticles by wet precipitation method”, Journal of Crystal Growth, Vol. 267, pp. 676-684, 2004; Lv et al., “Controlled synthesis of magnesium hydroxide nanoparticles with different morphological structures and related properties in flame retardant ethyolene-vinyl acetate blends”, Nanotechnology, Vol. 15, pp. 1576-1581, 2004; Zhang et al., “Surface treatment of magnesium hydroxide to improve its dispersion in organic phase by the ultrasonic technique”, Applied Surface Science, Vol. 253, pp. 7393-7397, 2007; and Shibli et al., “Development of phosphate inter layered hydroxyapatite coating for stainless steel implants”, Applied Surface Science, Vol. 254, pp. 4103-4110, 2008.

Optionally, the so-formed morphologies of porous material 36 can be stabilized by a so gel coating, such as MgO—CaO—FeO—ZnO—TaO. In embodiments, porous material 36 with a stabilizing sol gel coating can maintain its nano-structured morphology within a lumen for a longer time and thus provides a sustaining drug storage reservoir and releasing mechanism. In some embodiments, the stabilizing coating does not substantially blocks the pores of the porous material 36.

In embodiments, the surface of the so-formed porous material 36 can optionally be modified by including an oleic and stearic acid into the magnesium hydroxide. As is shown earlier in Scheme 1, the magnesium hydroxide surface is modified through an azeotropic distillation process, in which the drying and surface modification is realized in one pot. As a result, porous material can have a modified surface with a thickness of about 15 nm, which is hydrophobic and can facilitate absorption of hydrophobic drugs. Surface modification with oleic or stearic acid is also discussed in Li et al., Material Science and Engineering A 302, 452 (2007) and Lv et al., Colloids and Surfaces A: Physicochem. Eng. Aspects 97, 296 (2007).

In addition to the formation of and drug loading into the porous material 36, the non-porous solid material 90 exemplified in FIG. 8A or 8B can be deposited with a drug into the pores 38 by, for example, dip-coating, roll-coating, electrochemical coating, piezoelectric coating, and electrostatic or gas-assisted spray.

A stent with stent surface 30 can be used to deliver one or more therapeutic agents into a lumen. In embodiments, one or more therapeutic agents are applied to the stent in a manner that the one or more therapeutic agents fills one or more pores of the preform, a cross-sectional view of which is shown in FIG. 6. In particular embodiments, the one or more therapeutic agents are deposited without any non-therapeutic polymer or monomer carrier. The therapeutic agent can be deposited directly or by application in a suitable solvent. For example, the depositing can be effected by applying a potential difference, such as a few tens of millivolts to a few volts, between porous material 36 and the one or more therapeutic agents. In some embodiments, the one or more therapeutic agents are deposited into the porous material in discrete pores 38 by dip coating or spraying the preform with a solution of the one or more agents in a solvent, followed by drying to remove any solvent under low temperature conditions, e.g., ambient conditions. The loading can be facilitated by repeatedly dipping and/or spraying. In other embodiments, the one or more therapeutic agents are deposited to the porous material by a vapor deposition process, such as PLD. The one or more therapeutic agents, e.g., a drug, can be deposited by providing drug as a target material in the PLD apparatus.

In other embodiments, the one or more therapeutic agents can be deposited with a non-therapeutic polymer or monomer carrier. In embodiments, a polymer coating including one or more therapeutic agents can form a layer with an enhanced adhesion to stent surface 30 through the discrete anchoring sites formed by the portions of porous material 36 that extend beyond stent surface 30, a cross-sectional view of which is shown in FIG. 5. The stent is immersed into an electrolyte including an electropolymerizable monomer and a therapeutic agent or drug in an electrolytic cell, where the stent body 25 is utilized as a working electrode. A counter electrode and/or a reference electrode can be included in the cell. The therapeutic agent can be entrapped in a polymer matrix which grows onto the working electrode surface from the electrolyte containing the monomer and the therapeutic agent.

In embodiments, during the electropolymerization process, stent body 25 as the working electrode is given a positive potential, e.g., about a few hundred millivolts to about a few volts, and the monomer, e.g., pyrrole, is electrochemically oxidized at a polymerization potential giving rise to free radicals. In other embodiments, the stent as the working electrode is provided with a negative potential, e.g., about −100 millivolts to about −3 volts, and the monomer, e.g., 4-vinylpyridine, is electrochemically reduced to give rise to free radicals. These radicals are adsorbed onto or chemically bonded to the electrode surface and subsequently undergo a wide variety of reactions leading to the polymer network. The electropolymerization should preferably occur in a solution compatible for the drug to be incorporated into the polymer film in a suitable form. For example, organic solvents such as acetone, acetonitrile, tetrahydrofuran (“THF”), dimethyl formamide (“DMF”), and dimethylsulfoxide (“DMSO”), which can dissolve both the monomer and the drug, are suitable for producing the solution in which electropolymerization occurs. The growth of the corresponding polymer depends on its electrical character. If the polymer is electrically non-conducting, its growth is self-limited. Such films are very thin (about 10 nm to about 100 nm). In contrast, the growth of conductive polymers is virtually unlimited. In the latter case, the growth process is governed by the electrode potential and by the reaction time, which allows control of the thickness of the resulting film. The polymerization occurs locally and strictly on the electrode surface and the drug is entrapped in close proximity to the electrode surface. In addition, the combination of different conducting or non-conducting polymers allows the building of multilayer structures with extremely low thickness leading to different drug release profiles. The polymer film or coating can be generated by cycling the potential (“potentiodynamically”) or at a fixed potential (“potentiostatically”). The latter allows the more precise control of the film thickness and its growth.

In embodiments, only the pores of the stent are coated with a drug and/or polymer by exposing only the pores to the electrolyte, the result of which is shown in FIG. 7. For example, other surface areas can be selectively masked before the stent is immersed into the electrolyte, or only the surface region having the pores is immersed in the electrolyte while the others are not, or after the electrochemical deposition, coatings on the other surfaces are removed by, e.g., grinding, or laser ablation. In embodiments, different pores can be loaded with different drugs and/or polymers by, e.g., masking the desired (first) portions of the surface regions with pores so that drug will not be deposited in the masked (first) portions of the pores in a first electropolymerization process, and then removing the mask of those (first) portions so that the drug or a different drug will be deposited in this (first) portions in a second electropolymerization process. As a result, more diverse drug release profiles can be achieved.

In embodiments, a radiopaque material can be added in pores 38. Examples of radiopaque materials include BaO, Pt, Ta, tungsten, polymer modified iodine groups, or other suitable materials. The radiopaque material can have the form of a nanoparticle, nanorod, nanoribbon, nanotubes, or others. The radiopaque material can be loaded to pores 38 prior to the formation of porous material 36. For example, a radiopaque material can be loaded to pores 38 before the biodegradable metallic layer 82 is formed in the pores. The radiopaque material can also be incorporated during the preparation of porous material 36. The radiopaque material can be loaded after porous material 36 is formed. For example, the radiopaque can be loaded to porous material by physical vapor deposition process or electro-plating. Other suitable processes can also be used. The incorporation of a radiopaque material in a stent wall can facilitate locating and tracking the stent when it is delivered into lumen through radiation, e.g. X-ray and thus assist medication.

The terms “therapeutic agent,” “pharmaceutically active agent,” “pharmaceutically active material,” “pharmaceutically active ingredient,” “drug” and other related terms may be used interchangeably herein and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be nonionic, or they can be anionic and/or cationic in nature. Therapeutic agents can be used singularly, or in combination. Preferred therapeutic agents include inhibitors of restenosis (e.g., paclitaxel), anti-proliferative agents (e.g., cisplatin), and antibiotics (e.g., erythromycin). Additional examples of therapeutic agents are described in U.S. Published Patent Application No. 2005/0216074. In embodiments, the drug can be incorporated within the porous regions in a polymer coating. Polymers for drug elution coatings are also disclosed in U.S. Published Patent Application No. 2005/019265A. A functional molecule, e.g., an organic, drug, polymer, protein, DNA, and similar material can be incorporated into grooves, pits, void spaces, and other features of the stent.

Suitable polymers include, for example, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics such as polystyrene and copolymers thereof with other vinyl monomers such as isobutylene, isoprene and butadiene, for example, styrene-isobutylene-styrene (SIBS), styrene-isoprene-styrene (SIS) copolymers, styrene-butadiene-styrene (SBS) copolymers, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenerated polyalkylenes including polytetrafluoroethylene, natural and synthetic rubbers including polyisoprene, polybutadiene, polyisobutylene and copolymers thereof with other vinyl monomers such as styrene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions are also within the scope of the present invention. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment, the preferred polymer is polyacrylic acid, available as HYDROPLUS® (Boston Scientific Corporation, Natick, Mass.), and described in U.S. Pat. No. 5,091,205, the disclosure of which is hereby incorporated herein by reference. U.S. Pat. No. 5,091,205 describes medical devices coated with one or more polyiocyanates such that the devices become instantly lubricious when exposed to body fluids. In another preferred embodiment of the invention, the polymer is a copolymer of polylactic acid and polycaprolactone. Suitable polymers are discussed in U.S. Publication No. 2006/0038027.

In embodiments, the polymer is capable of absorbing a substantial amount of drug solution. When applied as a coating on a medical device in accordance with the present invention, the dry polymer is typically on the order of from about 1 to about 50 microns thick. Very thin polymer coatings, e.g., of about 0.2-0.3 microns and much thicker coatings, e.g., more than 10 microns, are also possible. Multiple layers of polymer coating can be provided. Such multiple layers are of the same or different polymer materials.

Any stent described herein can be dyed or rendered radiopaque by addition of, e.g., radiopaque materials such as barium sulfate, platinum or gold, or by coating with a radiopaque material. The stent can include (e.g., be manufactured from) metallic materials, such as stainless steel (e.g., 316L, BioDur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6Al-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003, and published as U.S. 2005/0070990; and U.S. application Ser. No. 11/035,316, filed Jan. 3, 2005, and issued as U.S. Pat. No. 7,727,273. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003, and published as U.S. 2004/0143317.

The stents described herein can be configured for vascular, e.g., coronary and peripheral vasculature or non-vascular lumens. For example, they can be configured for use in the esophagus or the prostate. Other lumens include biliary lumens, hepatic lumens, pancreatic lumens, urethral lumens.

The stent can be of a desired shape and size (e.g., coronary stents, aortic stents, peripheral vascular stents, gastrointestinal stents, urology stents, tracheal/bronchial stents, and neurology stents). Depending on the application, the stent can have a diameter of between, e.g., about 1 mm to about 46 mm. In certain embodiments, a coronary stent can have an expanded diameter of from about 2 mm to about 6 mm. In some embodiments, a peripheral stent can have an expanded diameter of from about 4 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. The stent can be balloon-expandable, self-expandable, or a combination of both (e.g., see U.S. Pat. No. 6,290,721).

All publications, patent applications, patents, and other references mentioned herein are incorporated by reference herein in their entirety.

Other features and advantages of the invention will be apparent from the following detailed description, and from the claims.

Other embodiments are in the following claims.

What is claimed is:

1. An endoprosthesis, comprising:
an endoprosthesis wall comprising
a surface comprising a metallic material defining a plurality of discrete pores, and
a porous magnesium oxide or iron oxide material disposed in one or more of the discrete pores.

2. The endoprosthesis of claim 1, wherein the endoprosthesis wall further comprising a non-porous solid material disposed in one or more of the discrete pores.

3. The endoprosthesis of claim 2, wherein the non-porous solid material comprises a bioresorbable ceramic.

4. The endoprosthesis of claim 3, wherein the bioresorbable ceramic comprises a material selected from the group consisting of hydroxyapatite, magnesium hydroxide, and calcium hydroxide.

5. The endoprosthesis of claim 2, wherein the porous magnesium oxide material and the non-porous solid material are disposed in different discrete pores.

6. The endoprosthesis of claim 2, wherein the porous magnesium oxide material and the non-porous solid material are disposed in the same discrete pores.

7. The endoprosthesis of claim 2, wherein the non-porous solid material comprises a matrix structure and contains a drug.

8. The endoprosthesis of claim 1, comprising a therapeutic agent in the porous magnesium oxide material.

9. The endoprosthesis of claim 1, comprising a polymer coating covering the surface layer, anchored by the porous magnesium oxide material disposed in one or more of the discrete pores.

10. The endoprosthesis of claim 1, wherein the magnesium oxide material extends beyond the surface of the well.

11. The endoprosthesis of claim 1, wherein the porous material has a morphology selected from a group consisting of nano-ribbon, nano-needle, nano-rod, rice grain, corn flakes, and cauliflower.

12. The endoprosthesis of claim 1, wherein the porous material comprises magnesium oxide or magnesium hydroxide.

13. The endoprosthesis of claim 1 wherein the porous material comprises a morphology-stabilizing sol gel coating.

14. The endoprosthesis of claim 13, wherein the sol gel coating comprises MgO—CaO—FeO—ZnO—TaO.

15. The endoprosthesis of claim 1, wherein the porous magnesium oxide material comprises a hydrophobicity enhancing agent.

16. The endoprosthesis of claim 15, wherein the hydrophobicity enhancing agent comprises an Oleic or Stearic acid.

17. The endoprosthesis of claim 1, wherein the metallic material is selected from a group consisting of stainless steel, Co—Cr alloy, MP35N, Nitinol, and PERSS.

18. The endoprosthesis of claim 9, wherein the polymer coating includes a therapeutic agent.

19. The endoprosthesis of claim 9, wherein the polymer is bioerodible.

20. The endoprosthesis of claim 1, wherein the endoprosthesis wall comprises an abluminal surface region and a luminal surface region, and the surface layer is in the abluminal surface region.

21. The endoprosthesis of claim 1, wherein the pore width is 500 nm or more.

22. The endoprosthesis of claim 1, wherein the oxide extends by about 0.1 micron or more above surface.

23. An endoprosthesis, comprising:
an endoprosthesis wall comprising
a surface layer comprising a metallic material and defining a plurality of discrete pores,
a porous ceramic material disposed in one or more of the discrete pores of the surface layer; and
a polymer coating over the surface layer.

24. A method for making an endoprosthesis, comprising:
forming on a surface of an endoprosthesis preform a plurality of discrete pores;
depositing a first material into one or more of the discrete pores;
corroding the first material to form a second material in the discrete pores; and
treating the second material in the discrete pores in a locally high alkaline environment to form a third material.

25. The method of claim 24, comprising corroding the first material by applying an electrolyte to the first material.

26. The method of claim 25, wherein the electrolyte applied to the first material comprises an aqueous NaCl solution or a solid polymer electrolyte.

27. The method of claim 24, comprising corroding the first material by anodically dissolving the first material.

28. The method of claim 27, comprising anodically dissolving the first material by applying a positive voltage to the first material.

29. The method of claim 24, comprising treating the second material by a water-alcohol solution.

30. The method of claim 24, further comprising coating the third material with a morphology-stabilizing sol gel coating.

31. The method of claim 24, further comprising loading a surface-modifying material comprising an oleic or stearic acid to the third material.

32. The method of claim 24, further comprising adding a radiopaque material into the third material.

33. The method of claim 24, wherein the first material comprises magnesium or magnesium alloy.

34. The method of claim 24, wherein the second material comprises magnesium salt, magnesium chloride, magnesium sulfate, or a combination thereof.

35. The method of claim 24, wherein the third material comprises magnesium oxide or magnesium hydroxide.

36. The method of claim 24, comprising depositing a solid non-porous material into one or more of the pores in which the first material is not deposited.

37. The method of claim 24, comprising depositing a solid non-porous material into one or more of the pores in which the first material is deposited.
